(19) United States
(12) Patent Application Publication
(10) Pub. No.: US 2014/0242168 A1
(43) Pub. Date: Aug. 28, 2014

(54) SWELLABLE COATED TABLET

(71) Applicant: LTS Lohmann Therapie-Systeme AG, Andernach (DE)

(72) Inventors: Christiane Schiller, Stralsund (DE); Knut Seidlitz, Greifswald (DE)

(73) Assignee: LTS LOHMANN THERAPIE-SYSTEME AG, Andernach (DE)

(21) Appl. No.: 14/131,014

(22) PCT Filed: Jul. 6, 2012
(86) PCT No.: PCT/EP2012/063235

§ 371 Date: May 14, 2014

(30) Foreign Application Priority Data
Jul. 7, 2011 (DE) 102011051653.0

(51) Int. Cl. A61K 9/28; A61K 31/635; A61K 31/522

(52) U.S. Cl. CPC A61K 9/2886; A61K 31/522; A61K 31/635; A61K 9/2893
USPC 424/468; 514/263.34; 514/158; 427/2.21

(57) ABSTRACT

The present invention provides perorally administrable drug release systems and processes for producing perorally administrable systems having a drug-containing core and a sheath which surrounds the core and comprises a swellable shell and an elastic coating which surrounds at least the shell, the sheath having at least one orifice.

The present application claims priority from PCT Patent Application No. PCT/EP20121063235 filed on Jul. 6, 2012, which claims priority from German Patent Application No. DE 10 2011 051 653.0 filed on Jul. 7, 2011, the disclosures of which are incorporated herein by reference in their entirety.

### FIELD OF THE INVENTION

The present invention relates to perorally administrable drug release systems having a continuous and long-lasting release of the drug present in the drug release system. The present invention relates especially to drug release systems having a prolonged residence time in the stomach.

It is noted that citation or identification of any document in this application is not an admission that such document is available as prior art to the present invention.

It is desirable for a number of drugs when, after peroral administration of the drug form containing this drug, they are released from the drug form homogeneously and over a prolonged period. Especially for drugs which serve for treatment of gastric disorders or which can be absorbed only over a relatively narrow absorption window in the upper region of the small intestine, long-lasting and homogeneous release in the stomach or gastrointestinal tract is therapeutically advantageous.

The primary clinical aim of employing gastroretentive systems, i.e. systems having a prolonged residence time in the stomach, is a long-lasting and continuous release of the drug in the stomach, and hence the possibility of local therapy in the stomach or absorption in the upper small intestine. Long-lasting, continuous drug absorption in the gastrointestinal tract requires continuous and homogeneous release of the drug from the stomach into the small intestine, since absorption of the drug takes place only in the small intestine. An optimal release system for drugs should therefore dwell in the stomach for as long as possible and continuously release the drug in the stomach. Only in this way can the therapeutic advantage of a delayed-onset, homogeneous and long-lasting effect be achieved. In addition, it is possible to improve the bioavailability of drugs for which there exists an absorption window in the upper small intestine.

The known drug release systems having prolonged dwell time in the stomach, which are supposed to bring about ongoing and homogeneous release of the drug present therein in the gastrointestinal tract can be classified on the basis of the mechanical principles underlying the prolonged dwell time thereof into at least one of the following groups:
Group I: drug release systems which have a lower density than water and float on top of the stomach contents;
Group II: drug release systems which have a higher density than water and sink into the body of the stomach;
Group III: expandable drug release systems which expand in the stomach, the size of which prevents passage through the pylorus;
Group IV: mucoadhesive drug release systems which adhere to the stomach wall;
Group V: drug release systems which inhibit the emptying of the stomach through pharmacodynamic or nutritional effects; and
Group VI: drug release systems of a particular form.

For the achievement of the primary clinical aim in the use of gastroretentive systems, release of the drug from the drug form with approximately 0th order kinetics is required. In drug form research, it is thus necessary to find a system which is both gastroretentive and has a constant release rate under the variable environmental conditions which exist in the stomach. This means that, for example, the composition of the medium in the stomach, the pH thereof and the volume thereof, and also the pressure stresses and the hydrodynamic conditions to which the drug form is exposed in the stomach, may have only a negligible influence at most, if any, on the release of the drug. The observation of the variable environmental conditions is important in that no better therapeutic benefit has been achieved to date solely through a prolonged dwell time of a drug form in the stomach. Only in the case of appropriate control of the blood accumulation of the active ingredient can gastroretentive administration systems be used profitably in a therapeutic context.

Published specification EP 0 779 807 A1 describes a tablet having an active ingredient-containing, erodable core and a largely erosion-resistant sheath. The sheath has at least one orifice and the core reaches the orifice at one of its ends. By virtue of the particular geometry of the core, the erosion area thereof increases with increasing distance from the orifice in the sheath. The increase in the erosion area counters the consequences of a lengthening diffusion pathway between erosion area and orifice with increasing erosion of the core, and pursues the purpose of homogeneous release of the active ingredient from the tablet.

An orally administrable administration form for controlled release of active ingredient in the stomach or the upper gastrointestinal tract is described in WO 01/56544 A2. This administration form comprises a core having a first polymer matrix in which the active ingredient has been dispersed, and a sheath of a second polymer matrix which fully surrounds the core, which is water-swellable and whose ratio of active ingredient to polymer is much less than that of the core. The administration form is notable for the thickness of the sheath which, as a diffusion barrier, controls the release of the active ingredient from the administration form. The release of the active ingredient from this administration form should have 0th order kinetics.

The problem on which the present invention was based was that of providing a gastroretentive drug release system with which blood accumulation of the drug to be administered can be controlled appropriately and which releases the drug at constant rate over a prolonged period.

The problem is solved by a perorally administrable drug release system having at least one drug-containing core and a sheath which surrounds the core and which comprises a swellable shell and an elastic coating which surrounds the shell, the sheath having at least one orifice. The present invention also provides a process for producing perorally administrable drug release systems having a drug-containing core and a sheath which surrounds the core and which comprises a swellable shell and an elastic coating which surrounds the shell, the sheath having at least one orifice. The invention further extends to the use of the perorally administrable drug release systems having a drug-containing core and a sheath which surrounds the core and which comprises a swellable shell and an elastic coating which surrounds the shell, the sheath having at least one orifice, for administration of at least one drug and for treatment of disorders.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

It is further noted that the invention does not intend to encompass within the scope of the invention any previously disclosed product, process of making the product or method of using the product, which meets the written description and enablement requirements of the USPTO (35 U.S.C. 112, first paragraph) or the EPO (Article 83 of the EPC), such that applicant(s) reserve the right to disclaim, and hereby disclose a disclaimer of, any previously described product, method of making the product, or process of using the product.

### SUMMARY OF THE INVENTION

In a first aspect, the present invention relates to a perorally administrable drug release system having a drug-containing core and a sheath which at least partly surrounds the core and which comprises a swellable shell and an elastic coating. The elastic coating surrounds the shell. The sheath further has at least one orifice through which the drug present in the drug-containing core can be released to the medium surrounding the drug release system.

### DETAILED DESCRIPTION OF EMBODIMENTS

It is to be understood that the figures and descriptions of the present invention have been simplified to illustrate elements that are relevant for a clear understanding of the present invention, while eliminating, for purposes of clarity, many other elements which are conventional in this art. Those of ordinary skill in the art will recognize that other elements are desirable for implementing the present invention. However, because such elements are well known in the art, and because they do not facilitate a better understanding of the present invention, a discussion of such elements is not provided herein.

The present invention will now be described in detail on the basis of exemplary embodiments.

In one embodiment, the inventive drug release system takes the form of a so-called core/shell tablet in the narrower sense, in which the tablet core is surrounded by a tablet shell. In a preferred embodiment, the tablet core apart from the at least one orifice in the sheath is fully surrounded by the tablet shell. This may mean that the drug-containing core in these embodiments extends as far as the at least one orifice in the shell, such that it is in contact with the medium which surrounds the drug release system. However, this can also mean that the orifice is present not just in the flexible coating but also in the swellable shell, such that a short channel through which the drug present in the core can be released to the medium surrounding the drug release system is formed in the sheath. Thus, even in the embodiments in which the core does not extend as far as the orifice in the elastic coating of the sheath, it can be in contact with the medium surrounding the drug release system.

In an alternative embodiment of the drug release system, the core is not surrounded essentially fully, i.e. apart from the at least one orifice in the sheath, by the shell, but embedded into the shell. This means that the drug-containing core is surrounded by the swellable material of the shell in the manner of a bath. In this configuration, the swellable shell and the drug-containing core are in contact with one another at the base surface of the core. The swellable shell and the drug-containing core are also in contact with one another at the lateral edges of the drug-containing core. However, the upper surface of the core is not covered by the shell. In this way, the upper surface of the core and the upper edge of the shell which takes the form of a bath can form a common surface which extends in the same plane. However, the drug release system in these embodiments can also be configured such that the upper surface of the core is lower or higher than the upper edge of the shell which takes the form of a bath. In a particularly preferred configuration, the upper end of the edge of the shell which takes the form of a bath extends inwards, such that the upper edge of the shell in the form of a bath encompasses the embedded core and thus enables better connection of core and shell. The aforementioned embodiments and configurations thereof are suitable especially for drug release systems in which the drug-containing core is surrounded by a semipermeable membrane, because improved drug release can be achieved thereby.

The drug-containing core is soluble or erodable in the gastric juice, such that the drug present in the core can be released to the gastric juice.

In one embodiment of the inventive drug release system, the drug-containing core is a pressed core. This means that the drug-containing core has been produced by pressing a powder or granular material consisting of the ingredients of the core, i.e. the drug and the pharmaceutical excipients which partly determine the physical, chemical and physicochemical properties of the core. In an alternative embodiment, the drug-containing core is a cast core. This means that the drug and any pharmaceutical excipients required are mixed with a molten matrix material and filled into casting moulds, where they solidify to form the cores.

In preferred embodiments, the core has the shape of a cylinder or of a prism, more preferably the shape of a prism having a triangular base area or an essentially triangular base area. This means that the core, in a top view, has three lateral edges connected to one another by three sides, each of which may be straight or of convex shape or concave shape. The triangular base area may be a base area corresponding to an equiangular or equilateral triangle. In side view of the core too, the upper side thereof and/or the lower side thereof may be straight, i.e. planar, concave or convex in shape. The triangular or essentially triangular base shape of the core offers the advantage of precise alignment of the core in the drug release system, such that at least one lateral edge of the core is positioned below an orifice in the sheath, in order that the core has contact via the at least one lateral edge with the medium surrounding the drug release system. In alternative embodiments, two of the three lateral edges or all three lateral edges are in contact with the medium surrounding the drug release system. This means that the drug release system has two or three orifices in the sheath surrounding the core, with the two or three lateral edges each in direct contact with one of the orifices.

The triangular or essentially triangular base shape of the core additionally offers the advantage, especially for the administration of a hydrophilic drug, that the decrease in the drug release because of the increasing diffusion distance between release front and orifice can be compensated for in an optimal manner by an increase in the release area, in order to achieve a constant release rate of the drug from the drug release system. Through the use of cores having concave and/or convex sides, particularly fine control of the drug release is possible.

The matrix material of the core in which the drug and optionally pharmaceutical excipients are distributed, dissolved or dispersed is soluble or erodable at least in the gastric juice. Preferred matrix materials for the core are selected from the group comprising polyethylene glycol, polyethylene oxide, block copolymers of ethylene oxide and propylene oxide, polyacrylates, polymethacrylates, polylactides, polyglycolides, polyalkylene oxide, polyvinyl alcohol, polyvinyl acetate, polyvinyl chloride, polyvinylpyrrolidone, macroglycerol fatty acid esters, celluloses, cellulose derivatives, starch, starch derivatives and mixtures thereof.

Basically, the core for the inventive drug release system may comprise any desired orally administrable drug. In particular embodiments, the core of the inventive drug release system may comprise at least one drug selected from the group consisting of ampicillin, digoxin, ketoconazole, fluconazole, griseofulvin, itraconazole, miconazole, metformin hydrochloride, vancomycin hydrochloride, captopril, lisinopril, erythromycin lactobionate, ranitidine hydrochloride, sertraline hydrochloride, ticlopidin hydrochloride, baclofen, amoxicillin, cefuroximaxetil, cefaclor, clindamycin, levodopa, doxifluridine, thiamphenicol, tramadol, fluoxetine hydrochloride, ciprofloxacin, bupropion, saquinavir, ritonavir, nelfinavir, clarithromycin, azithromycin, cinnarizine, ceftazidim, acyclovir, valaciclovir, ganciclovir, cyclosporin, paclitaxel, topiramat, oxpentifylline, bromocriptine mesylate, physostigmine, pyridostigmine bromide, rivastigmine, dihydroergotamine, propranolol, oxyprenolol, metropolol, timolol, sotalol, benazepril, cimetidine, furosemide, hydrochlothiazide, sulindac, diclofenac, flurbiprofen, ketoprofen, indomethacin, acetylsalicylic acid, dexamethasone, budesonide, beclomethasone, flucticasone, tioxocortol, oestradiol, theophylline, salbutamol, isosorbide dinitrate, isosorbide mononitrate, nifedipine, nimodipine, diltiazem, atenolol, cimetropium bromide, quinidine, verapamil, procainamide, lidocaine, methotrexate, tamoxifen, cyclophosphamide, mercaptopurine, etoposide, ergotamine, glibenclamide, 5-hydroxytryptamine, metkephamide, misoprostol, prednisolone, metoclopramide, pentoxifylline, diazepam and cisapride. It is also possible for a plurality of drugs to be present in one core.

The core of the inventive drug release system may additionally comprise one or more further pharmaceutical excipients. It is possible to add, for example, stabilizers, solubilizers, surfactants, fillers, plasticizers, hydrophilizing agents, pigments, substances for adjusting the pH, flow regulators, mould release agents and lubricants. The core may likewise include at least one hygroscopic substance. Preferably, the hygroscopic substance is selected from the group consisting of sodium chloride and calcium chloride.

In a particular embodiment of the inventive drug release system, the core is surrounded by a semipermeable membrane. The semipermeable membrane has at least one orifice, the at least one orifice in the semipermeable membrane being connected to the at least one orifice in the sheath, such that the core is in contact with the medium surrounding the drug release system. With the aid of the semipermeable membrane, the ingress of gastric juice to the core can be controlled.

Preferably, the semipermeable membrane is a coating formed from a material selected from the group consisting of cellulose acetate, cellulose triacetate, agar acetate, amylase triacetate, acetaldehyde dimethyl acetate, cellulose acetate methyl carbamate, cellulose acetate succinate, cellulose acetate dimethaminoacetate, cellulose acetate ethyl carbonate, cellulose acetate chloroacetate, cellulose acetate ethyloxalate, cellulose acetate-methylsulphonate, cellulose acetate butylsulphonate, cellulose ethers, cellulose acetate propionate, poly(vinyl methyl ether) copolymers, cellulose acetate-diethylaminoacetate, cellulose acetoacetate, cellulose acetate laurate, methyl cellulose, cellulose acetate p-toluenesulphonate, gum arabic triacetate, cellulose acetate with acetylated hydroxyethyl cellulose, hydroxylated ethylene-vinyl acetate, polymeric epoxides, copolymers of an alkylene oxide and alkyl glycidethyl ether.

Further excipients such as plasticizers or pigments may be present.

In this embodiment, the at least one orifice also extends through the semipermeable membrane to the core. This means that, in this embodiment, the semipermeable membrane also has an orifice.

This embodiment has particular advantages, especially for the administration of hydrophobic or sparingly water-soluble drugs, since it is possible with the aid of the semipermeable membrane to control the ingress of water or gastric juice to the drug-containing core and thus to prevent precipitation of the drug in the drug release system when the core erodes or goes into solution.

The classification into drugs of sparing water solubility and good water solubility is made according to the industry guidelines (Waiver of In Vivo Bioavailability and Bioequivalence Studies for Immediate-Release Solid Oral Dosage Forms Based on a Biopharmaceutics Classification System, U.S. Department of Health and Human Services, Food and Drug Administration (FDA), Center for Drug Evaluation and Research (CDER) of August 2000). This guidance defines the solubility as follows (page 2): The solubility class boundary is based on the highest dose strength of an immediate-release product that is the subject of a biowaiver request (drug substance for which a bioequivalence study can be omitted). A drug substance is considered highly soluble when the highest dose strength is soluble in 250 ml or less of aqueous media over the pH range of 1-7.5. The volume estimate of 250 ml is derived from typical bioequivalence study protocols that prescribe administration of a drug product to fasting human volunteers with a glass (about 8 ounces) of water.

The inventive drug release system has a sheath having a swellable shell and an elastic coating. The shell consists of a swellable material or a swellable material mixture comprising at least one swellable material. The swellable material is preferably a compound selected from the group consisting of cellulose polymers and derivatives thereof, polysaccharides and derivatives thereof, polyalkylene oxides, polyethylene glycols, chitosan, polyvinyl alcohols, xanthan gum, maleic anhydride copolymers, polyvinylpyrrolidones, starch and starch-based polymers, maltodextrins, poly(2-ethyl-2-oxazolines), poly(ethyleneamines), polyurethane hydrogels, crosslinked polyacrylic acids and derivatives thereof, hydrocolloids, alginates including sodium alginate and calcium alginate, and aluminas.

Preferred derivatives of cellulose polymers are hydroxymethyl cellulose, hydroxyethyl cellulose, hydroxypropyl cellulose, hydroxypropyl methyl cellulose, carboxymethyl cellulose and microcrystalline cellulose.

Particularly preferred examples of polyalkylene oxides which can be used in the inventive core/shell tablet are poly(ethylene oxide) and poly(propylene oxide). Poly(ethylene oxide) is a linear polymer of unsubstituted ethylene oxide. Poly(ethylene oxide) polymers having average molecular weights of about 300 000 g/mol or higher are preferred.

For the production of the shell layer, it is additionally possible to use further suitable excipients, for example flow regulators, lubricants or glidants, fillers, binders and/or separating agents. The fillers used may be starch derivatives, sugars such as sucrose or glucose, sugar substitutes such as xylitol or sorbitol.

Particular preference is given to using lactose or microcrystalline cellulose. The binders used may be polyvinylpyrrolidone; gelatin, methyl cellulose, ethyl cellulose, gum arabic, tragacanth, polyethylene glycol, starch derivatives. Usable glidants are magnesium stearate, calcium stearate, calcium behenate, glyceryl monostearate, stearic acid and salts thereof, waxes, finely divided silicon dioxide and hydrogenated vegetable fats. In addition, it is possible to use substances which can locally influence the pH, for example citric acid or algedrate. It is likewise possible for the shell layer to include at least one hygroscopic substance. The hygroscopic substance is preferably selected from the group consisting of sodium chloride and calcium chloride.

The advantage of a swellable shell is that the drug release system, after peroral administration, swells in the stomach as a result of the gastric juice present therein and thus increases in volume. Preferably, the drug release system, prior to administration, has a diameter in the region of at least 5 mm, preferably a diameter of at least 8 mm, and more preferably of at least 10 mm. The diameter of the drug release system is preferably not more than 20 mm, more preferably not more than 15 mm, and most preferably not more than 13 mm. Prior to administration thereof, the drug release system preferably has a height in the range from 6 to 11 mm.

After swelling in the stomach or in a release medium, the swelling index is determined. This is calculated from the change in mass of the tablet at time t (mₜ) after taking up the liquid in relation to the starting mass (m₀):

$$swelling index = \frac{{m}_{t} - {m}_{0}}{{m}_{0}} .$$

Preferably, the core/shell tablet after 4 hours has a swelling index of >2, more preferably >4.

As a result of this increase in size, the drug release system can only leave the stomach with a considerable time delay because it cannot pass the stomach outlet in the swollen state. The dwell time of such a drug release system in the stomach over which the medicament present in the drug release system can be released is distinctly prolonged compared to non-swellable administration forms.

The inventive drug release system also has an elastic coating which surrounds the shell except for the orifice(s) with which the sheath is provided. The task of this coating is to prevent premature erosion of the shell and disintegration of the drug release system. The coating has to be elastic in order that the drug release system can increase in volume in the stomach.

Preferably, the coating has a tensile strength in the range from 8 to 50 kg/mm², more preferably in the range from 8 to 40 kg/mm², in each case determined to DIN 53504 at 23° C./53% relative humidity. Preferably, the coating has an elongation at break in the range from 50 to 500%, more preferably in the range from 50 to 450%, in each case determined to DIN 53504 at 23° C./53% relative humidity. The choice of tensile strength and/or elongation at break within the aforementioned ranges ensures that the coating layer is sufficiently extensible and elastic so as not to tear when the shell layer expands, and simultaneously imparts sufficient stability to the core/shell tablet.

The coating layer is preferably based on a material selected from the group consisting of cellulose ethers, for example hydroxypropyl methyl cellulose or ethyl cellulose, cellulose esters, for example cellulose acetate, cellulose acetate butyrate or cellulose acetate propionate, polyacrylates and polymethacrylates, for example the products commercially available under the Eudragit® RS, Eudragit® RL or Eudragit® NE trademarks, polyvinyl derivatives, for example polyvinyl alcohol-polyether graft copolymers, polyvinyl acetates, for example the aqueous dispersion of polyvinyl acetate commercially available under the Kollicoat® SR 30 D trademark, copolymers of polymethyl vinyl ether and malonic acid or the ethyl, isopropyl and n-butyl esters thereof, for example the products commercially available under the Gantrez® AN trademark.

More preferably, the coating layer is based on at least one polyvinyl acetate. The polyvinyl acetate, which is preferably used in the form of an aqueous dispersion, can be stabilized by polymeric protective colloids. Suitable protective colloids are preferably polyvinylpyrrolidone (PVP), more preferably PVP K20 to K40, especially K30. The K value, which is also referred to as the intrinsic viscosity, is a classification customary in the polymer industry and correlates directly with the mean molar mass of the polymer. The K value is determined via viscosity measurements on polymer solutions and can be used in the industrial sector to determine the molar mass of polymers, since the K value, under constant measurement conditions in terms of solvent, solvent concentration and temperature, is dependent only on the mean molar mass of the polymers analysed. It is calculated via the relationship K value=1000·k by the Fikentscher equation, in which ηᵣ=relative viscosity (dynamic viscosity of the solution/dynamic viscosity of the solvent) and c=mass concentration of polymer in the solution in g/cm³. The Fikentscher equation is:

$$K = 1000 \cdot k = 1000 \cdot \frac{1.5 lg {η}_{r} - 1 \pm \sqrt{1 + ( \frac{2}{c} + 2 + 1.5 lg {η}_{c} ) \cdot 1.5 lg {η}_{r}}}{150 + 300 c}$$

It is thus possible to indirectly conclude the degree of polymerization and hence the chain length of the polymer from the K value.

The protective colloid is preferably used in an amount of 5 to 20% by weight, based on the amount of the vinyl acetate monomers.

In addition, however, alkylated, hydroxyalkylated or carboxyalkylated celluloses or starches are useful as protective colloids, for example hydroxypropyl cellulose, methyl cellulose, carboxymethyl starch, and also polyvinyl alcohols and vinylpyrrolidone-vinyl acetate copolymers.

A very particularly preferred material for production of the coating is an aqueous polyvinyl acetate dispersion containing 27% by weight of polyvinyl acetate, stabilized with 2.7% by weight of polyvinylpyrrolidone and 0.3% by weight of sodium dodecylsulphate. A material of this kind is commercially available, for example, under the Kollicoat®SR 30 D trade name from BASF, Ludwigshafen, Germany. The preferred coating additionally has a plasticizer content, more preferably of triethyl citrate (citric acid triethyl ester). Triethyl citrate should be present in the coating in an amount of between 2 and 5% by weight, based on the dry weight of the coating. A particularly preferred coating has a content of 87.09% by weight of polyvinyl acetate, 8.71% by weight of polyvinylpyrrolidone, 0.97% by weight of sodium dodecylsulphate and 3.23% by weight of triethyl citrate.

In addition, suitable excipients with which the properties of the coatings can be influenced may be added to the coating. Useful excipients include, for example, plasticizers, wetting agents or pigments. The plasticizers used may, for example, be esters such as triethyl citrate, triacetin, tributyl citrate, acetyltriethyl citrate, dibutyl tartrate, diethyl sebacate, dimethyl phthalate, diethyl phthalate, dioctyl phthalate, castor oil, sesame oil, glyceryl triacetate, glyceryl diacetate, higher alcohols, for example glycerol or propylene 1,2-glycol, or polyethers, for example polyethylene glycols. The plasticizers are especially suitable for establishing the desired elongation at break. Thus, it is possible through addition of plasticizers to distinctly increase the elongation at break of the coating layer. Suitable wetting agents are, for example, PEG-400 stearate, sorbitan monooleate and PEG-sorbitan monooleate. Suitable pigments are, for example, titanium dioxide and iron oxides. The use of such excipients can influence the properties of the coatings, since the mechanical properties such as flexibility, elasticity, brittleness and strength

Preferably, the coating has a thickness in the range from 1 mg/cm² to 10 mg/cm², more preferably in the range from 2 mg/cm² to 6 mg/cm².

An elastic coating, especially based on Kollicoat®SR 30 D, is sufficiently elastic to assure the swelling of the drug release system in the stomach but durable enough not to be destroyed by the mechanical stress in the stomach. The elastic coating keeps the shell in shape even in the swollen state. Without the elastic coating, the shell would disintegrate with increasing swelling.

In a second aspect, the present invention relates to a process for producing perorally administrable drug release systems having at least one drug-containing core and a sheath which surrounds the core and which comprises a swellable shell and an elastic coating which surrounds the shell, the sheath having at least one orifice. The drug-containing core may of course also contain a plurality of drugs.

The process according to the invention comprises the following steps:

a) producing a drug-containing core,
b) providing the drug-containing core with a swellable shell,
c) coating the swellable shell with an elastic coating, and
d) providing the sheath with at least one orifice.

In one embodiment of the process according to the invention, the core is produced by pressing a mixture of the ingredients of the core. For this purpose, the ingredients of the core are mixed with one another and the resulting mixture is pressed to cores. This method has the advantage that the ingredients need not be subjected to any thermal stress. For this purpose, the active ingredient-containing cores can be pressed from powder or granules on conventional tableting presses of the eccentric or rotary type. The production of the cores on an eccentric press can be effected with a shield-shaped die.

In an alternative embodiment of the process according to the invention, the core is produced by casting a melt consisting of the ingredients of the core. For this purpose, the matrix material of the core is melted and the drug and optionally additional excipients are dissolved or dispersed in the molten matrix material. The still-liquid or viscous mass is filled into casting moulds and allowed to solidify therein to give the cores, before the cores are removed from their casting moulds for further use. This embodiment has the advantage that the drug can be molecularly dispersed in the matrix material of the core, in order thus to achieve very substantially homogeneous release of the drug over the entire release time.

In a further, alternative embodiment, the drug-containing core is produced with the aid of a melt extrusion process. For this purpose, a mixture of at least one polymer, at least one active pharmaceutical ingredient and excipients is melted and shaped in a stable manner with the aid of an extruder, namely to the shape that the drug-containing core of the inventive drug release system is to have. The advantages of production of cores by means of melt extrusion are that the bioavailability of an active ingredient, for example of an active ingredient of sparing solubility in the gastric juice, can be improved. For instance, a solid dispersion stabilized by the polymer can be produced from a crystalline active ingredient. The size of the individual active ingredient particles can be reduced down to the molecular level, as a result of which the solubility thereof is increased tremendously. Further advantages are that melt extrusion is an economically viable, environmentally friendly and rapid, continuous process.

In an additional, optional step, the core, after production thereof, is surrounded by a semipermeable membrane before it is provided with the swellable shell. In order to enable the release of the drug from the core and the drug release system, the semipermeable membrane is also provided with at least one orifice. In a preferred embodiment of the process for producing the drug release system, the drug release system is provided with at least one orifice after the shell has been provided with the elastic coating. In this context, the sheath and any semipermeable membrane present are provided with the at least one orifice in one operation.

For the provision of the drug-containing core with a swellable shell, in a particular embodiment, some of the shell material of each and every tablet, preferably half of the shell material of each and every tablet, is introduced into the die of a tableting press, preferably of an eccentric press or of a core/shell tableting press which is capable of positioning the cores very precisely into the desired position in part-powder- or -granule-filled die openings and of pressing them with further powder or granules to give the core/shell tablet. The shell material may be in the form of granules or powder. After the core has been positioned onto the shell material introduced into the die and the rest of the shell material for each tablet has been introduced, the actual pressing operation is conducted. Core/shell tableting presses having the necessary precision in the core transfer are described, for example, in DE 40 25 484.

In another embodiment of the process, for the production of the drug release systems in which the drug-containing core is not essentially completely surrounded by the swellable shell material but is bonded to the swellable shell material, the entire shell material of a tablet is first introduced into the die of a tableting press, preferably of an eccentric press or a core/shell tableting press. Subsequently, a prefabricated core is positioned onto the shell material and the actual pressing operation is conducted.

After the drug-containing core has been provided with or bonded to the swellable shell, the resulting core/shell tablet is provided or coated with an elastic coating before the core/shell tablet is provided with one or more orifices. In this step, the sheath and any semipermeable membrane present are provided with the at least one orifice. According to the configuration of the drug release system, it is possible for only the elastic coating of the sheath to be provided with the at least one orifice, or both the elastic coating and the swellable shell. The at least one orifice extends through the sheath, through the elastic coating and optionally also the shell, and also through the semipermeable membrane optionally present, as far as the drug-containing core, such that the drug present in the core can be released through the at least one orifice into the medium surrounding the drug release system. The at least one orifice can be produced with the aid of a punch, of a drill, of a laser, or by cutting off, grating off or filing off. This means that the provision of the drug release system with at least one orifice is effected by punching, drilling, lasering, cutting off, grating off or filing off the material to be removed.

The present invention also extends to the use of the inventive drug release systems for peroral administration of at least one drug, especially for administration of a drug which is to display its therapeutic action in the stomach or upper small intestine, which has a relatively short half-life in the gastrointestinal tract and/or exists for a narrow absorption window in the upper small intestine, in order to achieve a long-lasting and homogeneous release of the drug in the stomach, preferably a release with 0th order kinetics.

Preferably, the drug release system dwells in the stomach for at least 6 hours, preferably for at least 8 hours and more preferably for at least 12 hours. The dwell time in the stomach is most preferably up to 16 hours. Once the core has dissolved or eroded, the swollen shell material can also escape from the drug release system through the at least one orifice, such that the shell material and the remaining coating, and also the semipermeable membrane optionally present, can be excreted via the patient's digestive tract.

The inventive drug release system succeeds in administering the drug in a particularly drug-conserving manner.

### BRIEF DESCRIPTION OF THE DRAWINGS

The invention is illustrated hereinafter by drawings and the working examples, the drawings and working examples serving merely for illustration but not restricting the invention.

FIG. 1A is a schematic sectional view of one embodiment of the inventive drug release system in top view. The drug release system 1 is in the form of a core/shell tablet comprising a core 2 and a shell 3 which essentially fully surrounds the core 2, i.e. apart from the orifice 5. In addition, the drug release system 1 comprises a coating 4 which essentially fully surrounds the shell 3. The drug release system 1 also has an orifice 5 which penetrates the shell 3 and the coating 4, such that the core 2 is in contact with the medium surrounding the drug release system 1.

FIG. 1B shows a schematic sectional drawing (longitudinal section) of a drug release system 1 also shown in FIG. 1A. The drug release system 1 is shown in the non-swollen state as present prior to peroral administration.

FIG. 1C shows the drug release system 1 which was shown in FIG. 1B in the swollen state as may be present in the stomach after peroral administration. In this case, the shell 3 has swollen through ingress of gastric juice and has thus increased in volume. Some of the core 2 has already gone into solution and has released the portion of the drug that was present in the portion of the core 2 which has already gone into solution. The face of the core 2 in contact with the medium is the release front 6 which moves increasingly away from the orifice 5 as a result of dissolution of the core 2 over the course of the release time, such that the distance between release front 6 and orifice 5 through which the drug is released from the system 1 increases.

FIG. 2 is a schematic drawing of a particular embodiment 10 of the inventive drug release system in top view. In the embodiment shown, the core 2 is embedded into the shell 3 such that the shell 3 surrounds the core 2 like a bath. In the embodiment shown, the core 2 is surrounded by a semipermeable membrane 7. Both the core 2 surrounded by a semipermeable membrane 7 and the shell 3 are encased by a common elastic coating 4. The drug release system 10 has an orifice 5 in the elastic coating 4 and the semipermeable membrane 7, through which the drug present in the core 2 can be released into the environment.

FIGS. 3 and 4 show diagrams with the release of particular drugs from particular embodiments of inventive drug release systems.

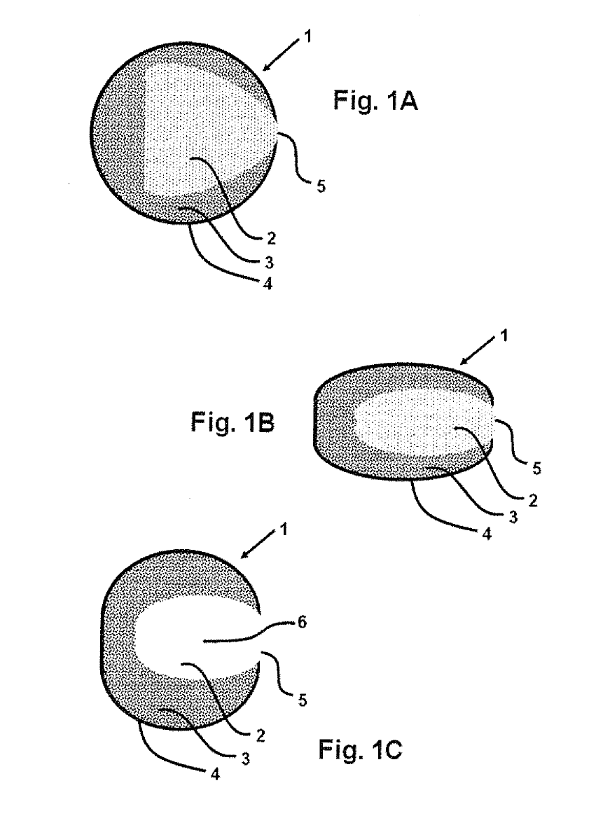

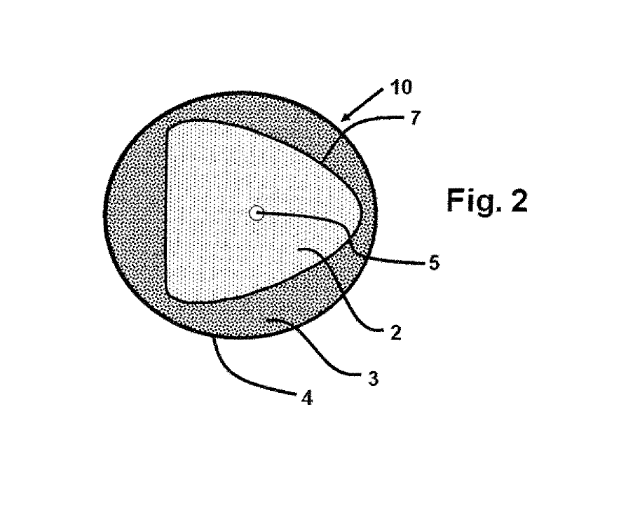

### OPERATIVE EXAMPLES

The subject matter of the present invention is elucidated in more detail below, using examples, without any intention that the subject matter of the invention should be confined to these exemplary embodiments.

### Example 1

### Production of a Swellable Core/Shell Tablet Comprising Caffeine

Core/shell tablets (drug release systems) having a core which comprised caffeine as a model active ingredient for readily water-soluble drugs were produced. Cast cores and pressed cores were produced in parallel. The cores had the following compositions:

TABLE 1
Composition of core/shell tablet cores comprising caffeine

| | Ingredient | Mass (g) | Mass (% by wt.) |
|---|---|---|---|
| Cast core | caffeine | 0.3 | 3.0 |
| | polyethylene glycol | 9.7 | 97.0 |
| | 1500 | | |
| | Sum: | 10.0 | 100.0 |
| Pressed core | caffeine | 0.3 | 3.0 |
| | microcrystalline | 9.6 | 96.0 |
| | cellulose | | |
| | magnesium stearate | 0.1 | 1.0 |
| | Sum: | 10.0 | 100.0 |

The cast cores were produced by melting the polyethylene glycol in a water bath and dispersing/dissolving the caffeine in the stated amount in the melt. The resulting mass was introduced into triangular casting moulds. After the mass had solidified, the cores were removed from the casting moulds.

To produce pressed cores, the ingredients in the stated amounts were mixed and granules were produced from the mixture. Subsequently, the granules were pressed on a tableting press by means of a shield-shaped die set to give triangular tablet cores. The die set had a diameter of 7 mm, based on the circle which connects the corners of a die of the shield-shaped die set (outer circle).

The shell too was produced in two different variants of differing composition. The compositions of the two variants are shown in the following table:

TABLE 2
Compositions of swellable shell layers

| | Ingredient | Mass (g) | Mass (% by wt.) |
|---|---|---|---|
| Variant | sodium alginate | 290.00 | 72.50 |
| | lactose | 100.00 | 25.00 |
| | monohydrate | | |
| | silicon dioxide | 8.00 | 2.00 |
| | magnesium | 2.00 | 0.50 |
| | stearate | | |
| | Sum: | 400.00 | 100.00 |
| Variant 2 | hypromellose | 12.65 | 5.06 |
| | polyethylene oxide | 163.075 | 65.23 |
| | sodium chloride | 73.675 | 29.47 |
| | magnesium | 0.60 | 0.24 |
| | stearate | | |
| | Sum: | 250.00 | 100.00 |

The ingredients of the shell were granulated in an ethanolic solution of polyvinylpyrrolidone, dried and then screened, and pressed together with the cores produced beforehand to give biconvex core/shell tablets having a diameter of 11 mm. For this purpose, half of the shell granules for each core/shell tablet were introduced into the die of an eccentric press. For each core/shell tablet, one core was positioned onto the initial charge of shell granules, the second half of the shell granules for each tablet was introduced above the respective core, and the pressing operation was conducted.

The core/shell tablets were subsequently coated with about 3 mg/cm² of a tablet coating. The coating had the following composition:

TABLE 3
Composition of the coating for core/shell tablet

| Ingredient | Mass (g) | Mass (% by wt.) |
|---|---|---|
| Kollicoat ®SR 30 D | 2.00 | 19.96 |
| acetone | 8.00 | 79.84 |
| triethyl citrate | 0.02 | 0.20 |
| Sum: | 10.02 | 100.00 |

Kollicoat®SR 30 D is the trade name for an aqueous polyvinyl acetate dispersion containing 27% by weight of polyvinyl acetate and stabilized with 27% by weight of polyvinylpyrrolidone and 0.3% by weight of sodium dodecylsulphate. Kollicoat®SR 30 D is commercially available from BASF, Ludwigshafen, Germany.

At the point with the shortest distance of a corner of the core from the coating of the core/shell tablet, a punch (diameter 2 mm) was used to punch a hole in the coating and the shell, such that the core is in contact with the medium surrounding the core/shell tablet.

The release rate for caffeine was determined with a paddle stirrer release apparatus for core/shell tablets having a pressed core and a shell having a composition according to variant 2. For this purpose, 1000 ml of an artificial gastric fluid (Simulated Gastric Fluid sine Pepsin) at a temperature of 37±0.5° C., pH 1.2, were used at a stirrer speed of 75 revolutions per minute. 3 core/shell tablets were examined in each case. One, 2, 3, 4, 5, 6, 7, 8 and 24 hours after the start of the experiment, samples of the artificial gastric fluid were taken and the caffeine content thereof was determined. Over the course of the release experiments, it was observed that the size of the core/shell tablets rose from initially diameter 11 mm to diameter about 25 mm.

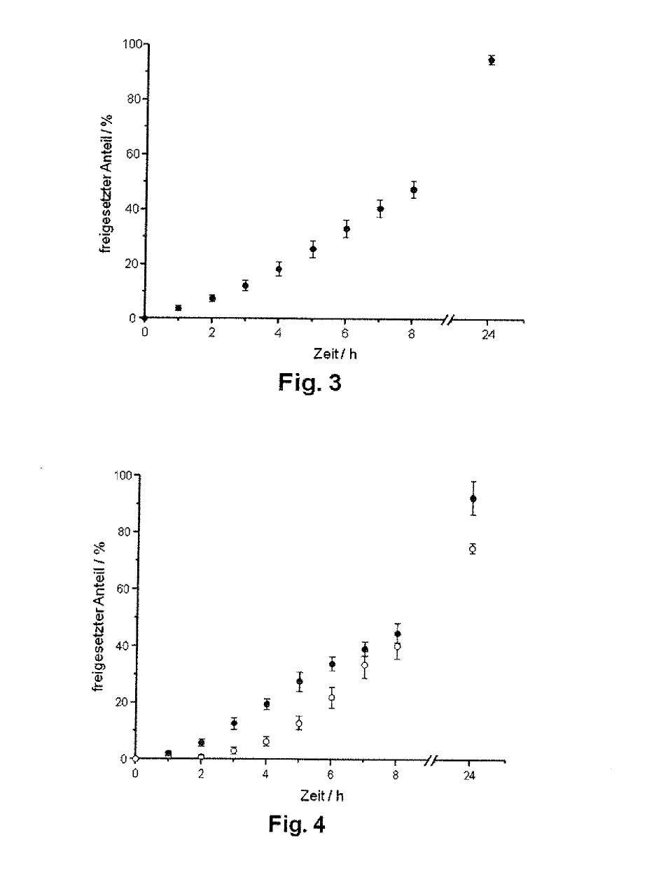

The results of the release of caffeine from the core/shell tablet are shown as a graph in FIG. 3. It becomes clear from the graph that caffeine was released homogeneously and at constant rate from the core/shell tablets.

### Example 2

### Production of a Swellable Core/Shell Tablet Comprising Furosemide

Two kinds of core/shell tablets (drug release systems) were produced, the cores of which comprised furosemide (4-chloro-2-furfurylamino-5-sulphamoyl-benzoic acid) as the drug. The composition of the cores is stated in Table 4.

TABLE 4
Composition of core/shell tablet cores comprising furosemide

| | Ingredient | Mass (g) | Mass (% by wt.) |
|---|---|---|---|
| Variant 1 | furosemide | 10.00 | 10.00 |
| | polyethylene oxide | 44.75 | 44.75 |
| | Pluronic ®F 68 | 44.75 | 44.75 |
| | magnesium stearate | 0.50 | 0.50 |
| | Sum: | 100.0 | 100.0 |
| Variant 2 | furosemide | 30.00 | 20.00 |
| | polyethylene oxide | 29.49 | 19.66 |
| | Gelucire ®50/13 | 82.005 | 54.67 |
| | sodium chloride | 8.505 | 5.67 |
| | Sum: | 150.00 | 100.00 |

Pluronic® F 68 is the trade name for a block copolymer of ethylene oxide and propylene oxide which is commercially available from BASF, Ludwigshafen, Germany, and has the following physical properties:

TABLE 5
Physical properties of Pluronic ®F 68

| Average molecular weight | 8400 | |
|---|---|---|
| Density (77° C./25° C.) | 1.06 | |
| Viscosity (cps at 77° C.) | 1000 | |
| Melting point | 52° | C. |
| Surface tension (0.1% in water) | 50 dynes/cm at 25° C. | |
| HLB | >24 | |
| Solubility in water <10% | | |
| Cloud point (0.1% in water) | >100° | C. |

Gelucire®50/13 is a nonionic, water-dispersible detergent composed of PEG esters, a small glyceride fraction and free PEG. Gelucire®50/13 (Stearyl Macrogolglyceride Ph. Eur.; mono-, di- and triglycerides, and mono- and diesters of polyethylene glycol) has CAS No. 121548-05-8 and is commercially available, for example, from Gattefossé, Lyons, France.

For the production of the cores according to variant 1, the stated amount of Pluronic® F 68 was comminuted in a mortar, screened through a 0.8 mm hand-held sieve and then mixed with furosemide, polyethylene oxide and magnesium stearate. From this mixture, the cores were pressed on an eccentric press with a shield-shaped die (outer circle diameter: 7 mm). The cores comprising Gelucire®50/10 were produced by a homogeneous distribution of the active ingredient in the molten Gelucire base. After solidification, the mixture was comminuted, screened through a 0.8 mm hand-held sieve and then mixed with polyethylene oxide and sodium chloride. From this mixture, cores were pressed on an eccentric press having a shield-shaped die (outer circle diameter: 7 mm). Subsequently, the cores were provided with a semipermeable coating based on cellulose acetate, which led to a semipermeable membrane. The coating material for the coating of the cores had the following, specified in Table 6:

TABLE 6
Composition for the core coating of cellulose acetate

| Ingredient | Mass (g) | Mass (% by wt.) |
|---|---|---|
| cellulose acetate | 4.00 | 4.00 |
| polyethylene glycol | 2.00 | 2.00 |
| triacetin | 0.14 | 0.14 |
| 2-propanol | 9.39 | 9.39 |
| acetone | 84.47 | 84.47 |
| Sum: | 100.00 | 100.00 |

Two kinds of core/shell tablets were produced. For one kind, half of the shell granules for each shell tablet were first introduced into the die of an eccentric press. For each core/shell tablet, a core was positioned onto the initial charge of shell granules, the second half of the shell granules for each tablet were introduced above the respective core and the pressing operation was conducted. For the other kind of core/shell tablets, all of the shell granules were introduced into the die of an eccentric press and the core was positioned centrally onto the initial charge of granules and then the pressing operation was conducted.

The material for the shell had the composition specified for variant 2 in Table 2. The ingredients of the shell were granulated in an ethanolic solution of polyvinylpyrrolidone, dried and then screened, and pressed together with the cores produced beforehand to give biconvex core/shell tablets having a diameter of 11 mm.

The core/shell tablets were subsequently coated with about 3 mg/cm² of a tablet coating according to Table 3. Subsequently, in the case of the core/shell tablets in which the core was surrounded by the shell on all sides, a punch (diameter 2 mm) was used to punch a hole into the coating, the shell and the semipermeable core coating at the point with the shortest distance of a corner of the core from the coating of the core/shell tablet, such that the core can come into contact with the medium surrounding the core/shell tablet and the furosemide present in the core can be released. In the case of the core/shell tablets in which the core has been positioned on the initial charge of granules and the tablet has then been pressed, such that the upper face of the core was in direct contact with the coating, the punch (diameter 2 mm) was used to punch a hole in the coating and the semipermeable core coating in the centre of the region in which the core was in contact with the coating, such that the core can come into contact with the medium surrounding the core/shell tablet and the furosemide present in the core can be released.

The release rate for furosemide was determined with a paddle stirrer release apparatus for the core/shell tablets with a core according to variant 2, both for the kind of core/shell tablet in which the core was present centred within the shell and for the kind in which the core was arranged atop the shell. For this purpose, 1000 ml of an acetate buffer at a temperature of 37±0.5° C., pH 4.5, were used at a stirrer speed of 75 revolutions per minute. 3 core/shell tablets were examined in each case. One, 2, 3, 4, 5, 6, 7, 8 and 24 hours after the start of the experiment, samples of the acetate buffer were taken and the furosemide content thereof was determined. Over the course of the release experiments, it was observed that the size of the core/shell tablets rose from initially diameter 11 mm to diameter of about 25 mm.

The results for the release of furosemide from the core/shell tablets are shown as a graph in FIG. 4. It becomes clear from the graph that furosemide was released homogeneously and at constant rate, with a discernible latency phase in which no furosemide was detectable in the acetate buffer for the core/shell tablets in which the core was centred within the shell (open circles). In the release experiment with the kind of core/shell tablets in which the core was bonded atop the shell material (closed circles), no such latency phase was observed.

While this invention has been described in conjunction with the specific embodiments outlined above, it is evident that many alternatives, modifications, and variations will be apparent to those skilled in the art. Accordingly, the preferred embodiments of the invention as set forth above are intended to be illustrative, not limiting. Various changes may be made without departing from the spirit and scope of the inventions as defined in the following claims.

1. A perorally administrable drug release system comprising a
a drug-containing core, and
a sheath which at least partly surrounds the core, wherein the sheath comprises a swellable shell and an elastic coating which surrounds the shell, and wherein the sheath further comprises at least one orifice.

2. The drug release system according to claim 1, wherein the core apart from the at least one orifice in the sheath is fully surrounded by the shell.

3. The drug release system according to claim 1, wherein the core is embedded into the shell such that one side of the core is in contact with the elastic coating.

4. The drug release system according to claim 1, wherein the core is surrounded by a semipermeable membrane having at least one orifice.

5. The drug release system according to claim 4, characterized in that the at least one orifice in the semipermeable membrane is connected to the at least one orifice in the sheath.

6. The drug release system according to claim 1, wherein the core is in the form of a prism having a triangular or essentially triangular base area.

7. The drug release system according to claim 1, wherein the elastic coating has a content of 87.09% by weight of polyvinyl acetate, 8.71% by weight of polyvinylpyrrolidone, 0.97% by weight of sodium dodecylsulphate and 3.23% by weight of triethyl citrate.

8. A process for producing a perorally administrable drug release system having at least one drug-containing core and a sheath which at least partly surrounds the core, wherein the sheath comprises a swellable shell and an elastic coating which surrounds the shell, and wherein the sheath further comprises at least one orifice, comprising the following steps:
a) producing the drug-containing core;
b) providing the drug-containing core with the swellable shell;
c) coating the swellable shell with the elastic coating; and
d) providing the sheath with at least one orifice.

9. The process according to claim 8, wherein the drug-containing core is surrounded by a semipermeable membrane before it is provided with the swellable shell.

10. The process according to claim 8, wherein the drug-containing core is produced by pressing a mixture of the ingredients of the core, by casting a melt consisting of the ingredients of the core, or by extruding a melt consisting of the ingredients of the core.

11. The process according to claim 8, wherein the drug release system is provided with at least one orifice by punching, drilling, lasering, cutting off, grating off or filing off the material to be removed.

12. A method comprising utilizing the drug release system according to claim 1 for peroral administration of a drug for long-lasting and homogeneous release of the drug in the stomach.

13. A process for producing a perorally administered drug release system as claimed in claim 1 comprising:
a) producing the drug-containing core;
b) providing the drug-containing core with the swellable shell;
c) coating the swellable shell with the elastic coating; and
d) providing the sheath with at least one orifice.
